(19) United States
(12) Patent Application Publication
(10) Pub. No.: US 2018/0235525 A1
(43) Pub. Date: Aug. 23, 2018

(54) DRIVER CIRCUIT

(71) Applicant: KONINKLIJKE PHILIPS N.V., EINDHOVEN (NL)

(72) Inventor: Pieter Gerrit BLANKEN, NUENEN (NL)

(73) Assignee: KONINKLIJKE PHILLIPS N.V., EINDHOVEN (NL)

(21) Appl. No.: 15/753,364

(22) PCT Filed: Aug. 30, 2016
(86) PCT No.: PCT/EP2016/070428

§ 371 Date: Feb. 19, 2018

(30) Foreign Application Priority Data
Sep. 14, 2015 (EP) 15185039.3

(51) Int. Cl. A61B 5/1455; H02M 3/156; H05B 33/08

(52) U.S. Cl. CPC A61B 5/14551; H02M 3/156; A61B 2560/0209; H05B 33/0824; H05B 33/0815

(57) ABSTRACT

The invention describes a pulse oximeter driver circuit (1) realized to drive a load (12) comprising at least a red LED light source (SR) and an infrared LED light source (SIR), which driver circuit (1) comprises a current regulator (11) realized to provide LED current (I_{load}) to the LED light sources (S_{R}, S_{IR}); a power converter (10) realized to provide a DC output voltage (Uₜₒₜₐₗ) comprising a current regulator voltage (U₁₁) and a load voltage (U_{load}); and a feedback means (13A, 13B, 13C) realized to obtain a measure (U_{FB}) of the instantaneous voltage drop (U₁₁) across the current regulator (11) and to report the voltage drop measure (U_{FB}) to the power converter (10). The invention further describes a pulse oximeter device (100), and a method of driving a pulse oximeter load (12).

### FIELD OF THE INVENTION

The invention describes a driver circuit for a pulse oximeter, a pulse oximeter arrangement, and a method of driving a pulse oximeter load.

### BACKGROUND OF THE INVENTION

A pulse oximeter is used to obtain an estimation of the oxygen saturation level of a patient's blood. Large pulse oximeter arrangements as discloses in US 2007/0149865 A1 have multiple sensors and can monitor oxygen saturation level simultaneously at several sites. A sensor can be realized as a small device that fits over the patient's finger, earlobe, toe etc., comprising a red LED and an infrared LED arranged on one inner side of the device, and a light detector arranged for example on the opposite side. Other designs are also possible. Light from the LEDs passes through the finger. Some of the light is absorbed and the remainder is detected. A calibrated pulse oximeter can deliver a good estimation of the oxygen saturation level of the patient's blood as it passes through arteries and veins of the tissue enclosed by the oximeter. The calculation of the blood oxygen saturation level is based on the ratio of the absorbance values of red and infra-red light, and makes use of the fact that oxygenated haemoglobin absorbs more infrared light than red light, while deoxygenated haemoglobin absorbs more red light than infrared light. To measure the oxygen saturation, the red LED and the infrared LED are alternately switched on over precise time intervals, each with an accurately determined low-noise current level.

In a vital signs monitoring scenario, a patient's blood oxygen saturation level may be monitored over a long period of time, for example over several hours, and for this reason a pulse oximeter may be realized as a compact battery-operated device that allows a patient some freedom of movement while wearing it. Power consumption is therefore an important design consideration, whereby the total power consumption is influenced by various factors. An LED driver for the red and infrared LEDs is generally mounted in a housing for electronics, and the driver, control circuitry, LEDs and light sensors can be interconnected using wires, cables, connectors etc. The pulse oximeter electronics may also be equipped with input and output filters and/or protection circuitry. Extension cables may be required between a sensor (LEDs, photo detector) and sensor electronics, and such extension cables may have a length in the range of a few meters. The room-temperature forward-bias voltages of the red and infrared LEDs in a pulse oximeter can be about 2.0 V and 1.5 V respectively at a typical operating current of 40 mA. Resistive voltage drops occur in output filters, circuit protection measures, flex cables, connectors, cables and any extension cables. Any voltage drops across (parasitic) series resistances in filters, protection circuitry, cables and connectors may be significant with respect to the LED voltage drop, and can vary from sensor to sensor, for example depending on the length of the cable or extension cable. A total series resistance of about 5.0Ω can lead to a resistive voltage drop of 0.2 V at 40 mA. The LED driver must generate the drive current at voltages exceeding the forward-bias voltage drop of the LED, and an accurately functioning current regulator is generally implemented for this purpose. It may also be necessary to take into account any sensor originating from a different manufacturer. A worst-case total series resistance can exceed 45Ω, which corresponds to a resistive voltage drop exceeding 1.8 V at a typical operating current of 40 mA. Therefore, a worst-case sum of LED forward-bias voltage and resistive voltage drop—i.e. the worst-case load voltage that must be supplied by the LED driver—can approach a maximum allowable load voltage of 4.0 V for a typical operating current of 40 mA. This maximum voltage of 4.0 V has been established over the years on the basis of experience gathered in situations in which vital signs monitoring is critical even under adverse conditions, for example in an ambulance at an accident site. Using some known types of LED driver topology, a maximum allowable load voltage of 4.0 V and a minimum voltage drop of 0.5 V across the analogue current regulator results in power consumption of at least 180 mW, regardless of whether high or low series resistances are present, and regardless of whether the red or the infra-red LED is being driven. For a battery-powered vital signs monitoring device, this is an unfavorably high level of power consumption. Batteries that deplete quickly must be replaced more often, adding to the cost of operation.

Therefore, it is an object of the invention to provide an improved driver with reduced power consumption that extends the battery life of a pulse oximeter. It is also an object of the invention to increase the worst-case load voltage without adversely affecting power efficiency or battery life.

### SUMMARY OF THE INVENTION

The object of the invention is achieved by the pulse oximeter driver circuit of claim 1; by the pulse oximeter device of claim 13; and by the method of claim 14 of driving a pulse oximeter load.

According to the invention, the pulse oximeter driver circuit is realized to drive a load comprising at least a red LED light source and an infrared LED light source and a switching module for alternately switching the red LED light source and the infrared LED light source, and comprises an analogue current regulator or linear current regulator realized to provide LED current to the LED light sources; and a power converter realized to provide a DC output voltage comprising a current regulator voltage and a load voltage. The pulse oximeter driver circuit according to the invention further comprises a feedback means realized to obtain a measure of the instantaneous voltage drop across the current regulator and to report the voltage drop measure to the power converter such that the driver circuit responds to a shift in voltage levels across the load when one LED light source is switched off and the other LED light source is switched on by the switching module.

As explained above, the forward voltage across an infrared LED is lower than the forward voltage across a red LED. A characteristic feature of a pulse oximeter load is that its red LED light source and infrared LED light source are always driven separately, i.e. one of the light sources is “off” while the other is “on”, so that the different LED light sources are never active at the same time. The linear current regulator delivers a constant controlled current to the active LED light source. In the context of the invention, a measure of the instantaneous voltage drop across the current regulator is “reported” to the power converter, and this may be taken to mean that the feedback means provides any suitable indication to the power converter that a change in the instantaneous voltage drop across the current regulator is occurring or has occurred. Such a change in voltage drop can be a transition between a first load voltage level and a second load voltage level when one LED light source is turned off, and the other is turned on, since the LED light sources have different forward voltages.

Generally, in a device such as a pulse oximeter, the red LED and infrared LEDs are switched essentially simultaneously, i.e. when one is switched off, the other is switched on. An advantage of the pulse oximeter driver circuit according to the invention is that the feedback means allows the driver circuit to respond to a shift in voltage levels across the load when one LED light source is switched off and the other is switched on. This “reported” information can be used by the driver circuit to regulate the power delivered to the load so that the load only consumes as much power as it requires at any one interval. The resulting low power consumption makes the inventive driver circuit particularly favorable for use in a battery-powered pulse oximeter.

According to the invention, the pulse oximeter device comprises terminals for connecting to a DC power supply; a red LED light source and an infrared LED light source; a switching module for alternately switching the red LED light source and the infrared LED light source; and a driver circuit according to the invention for driving the load comprising at least the red LED light source, the infrared LED light source and the switching module.

An advantage of the pulse oximeter device according to the invention is that the power-efficient driver circuit provides additional voltage “headroom”, allowing a relaxation on the requirements regarding worst-case resistance values of internal connections such as flex boards, and accessories such as external adapters, extension cables, connectors, etc. This in turn lowers design and manufacturing costs, particularly regarding the accessories, so that the inventive pulse oximeter device is very attractive from the point of view of power consumption and design costs.

According to the invention, the method of driving a pulse oximeter load consisting of at least a red LED light source and an infrared LED light source comprises at least the steps of providing a current regulator to deliver drive current to the LED light sources; providing a switched-mode converter to deliver an output voltage comprising a load voltage and an essentially constant or steady-state voltage drop across the current regulator; and obtaining a measure of the instantaneous voltage drop across the current regulator and reporting the voltage drop measure to the power converter.

When an LED light source is being driven during any one interval, the voltage drop across the current regulator (i.e. the difference in voltage between an input node and an output node of the current regulator) is at an essentially constant steady-state level. The method according to the invention makes use of the fact that different colored LEDs have different forward voltages, so that at the instant of switching one off and the other on, the voltage drop across the current regulator will deviate measurably from its steady-state level. An advantage of the method according to the invention is that it allows a driver circuit to respond to the different voltage levels arising from the different forward voltages of the red and an infrared LED light sources, so that—compared to prior art drivers—less power is consumed during the infrared interval, so that the overall power consumption of the driver circuit can be reduced, allowing a relaxation of the worst-case resistance requirements of internal connections and external connections and accessory components.

The dependent claims and the following description disclose particularly advantageous embodiments and features of the invention. Features of the embodiments may be combined as appropriate. Features described in the context of one claim category can apply equally to another claim category.

In the following, without restricting the invention in any way, it may be assumed that the driver is for use in a pulse oximeter device which is to be driven from a DC power supply, for example a 5.0 V DC supply. The switching module for alternately switching the red LED light source and the infrared LED light source can be realized on the basis of a switch matrix, which can be connected to the LEDs in the pulse oximeter by means of a cable connection, as will be known to the skilled person. An “LED light source” in the context of the invention may comprise one or more LEDs of a certain color, arranged in a device such that the light emitted by the LEDs is directed into human tissue, for example a patient's finger. Such a device may be assumed to also comprise a photo-detector arrangement for detecting any light that manages to pass unabsorbed through the tissue. It may be assumed that such a photo-detector arrangement can deliver its measurements to a suitable analysis unit.

Preferably, the current regulator is accurate, and is realized to deliver an essentially constant LED drive current, for example an LED drive current of 40 mA. In order for the current regulator to deliver a current with an accurate level to the load, the voltage drop across the current regulator should exceed a minimum level. Depending on the actual design of the regulator, such a minimum level is usually in the range of 0.2 V 0.6 V. However, when the voltage drop is higher, for example 3.2 V, the current regulator is still capable of delivering an accurate output current to the load, but the power dissipation in the regulator is much higher. Energy is therefore being wasted. In the drive circuit according to the invention, the voltage drop across the regulator is kept close to its allowable minimum value in order to minimize power dissipation.

Since the current regulator provides the LED drive current, this unit is directly connected to the load, which in turn comprises the red and infrared LEDs. Therefore, in a particularly preferred embodiment of the invention, the feedback means is connected between nodes of the current regulator and a feedback input port of the power converter. As mentioned above, the voltage drop across the accurate current regulator is kept essentially constant by the method according to the invention, regardless of which LED light source is being driven. Therefore, a change in voltage drop across the current regulator can provide an indication relating to the switching or toggling of the LED light sources. For example, the voltage across the current regulator initially tends to rise when the switch matrix turns off the red LED and turns on the infra-red LED, since the forward voltage of the infrared LED is lower than the forward voltage of the red LED. In other words, the driver circuit according to the invention controls the power converter so that this always delivers only the required total voltage at its outputs according to the type of LED light source that is “on”.

In a preferred embodiment of the invention, the feedback means is realized as a module comprising a first input node and a second input node, and is realized to convert a voltage difference across those input nodes into an output voltage that is connected to a feedback input port of the power converter. Generally, a power converter such as a switched-mode power converter is used to provide a constant output voltage level. To this end, most power converters of that type also feature a feedback input port to which an attenuated version of the output voltage is fed back. On the basis of the voltage across this feedback input port, the power converter can determine whether it needs to adjust its output voltage level. In the following, without restricting the invention in any way, the term “feedback input port of the converter” can be understood to mean a dedicated input pin of an integrated circuit and a negative supply terminal. When applying a feedback voltage to such a pin, the feedback means and the converter preferably share a common negative terminal. The term “port” as used in the context of the invention, in which positive DC voltages are being interpreted, may be understood to include a positive node and a common negative node such as the negative supply terminal of the DC power supply.

A first voltage-adding topology of the inventive driver circuit comprises a series connection of the power converter, current regulator and pulse oximeter load between the positive and negative supply terminals. In a second voltage-adding topology, the power converter, pulse oximeter load and current regulator are series-connected in that order. In each case, the instantaneous voltage drop across the current regulator is used to regulate the output voltage of the power converter, as will be explained in the following. Preferably, the feedback module comprises an amplifier circuit portion for converting the differential input voltage to a differential output voltage for connecting to the feedback input port of the power converter. Preferably, the voltage gain of the feedback module is determined on the basis of the target voltage drop across the current regulator and the internal voltage reference of the power converter which determines its setpoint value. For example, the voltage gain of the feedback module may be unity in the case of a power converter with a setpoint value of 0.6 V, and a current regulator voltage drop of about 0.6 V. Alternatively, the gain of the feedback module may be greater than 1.0. For example, in the case of a power converter with a setpoint value of 1.25 V, and a target voltage drop of 0.52 V across the current regulator, the feedback module can preferably be realized to have a voltage gain in the region of 2.4.

The feedback module can be realized in any number of ways that will be familiar to the skilled person. For example, the feedback module can comprise a level-shifting amplifier based on an op-amp, input resistor and transistor to convert a voltage difference across the input port into a current, which in turn is converted by means of a second resistor to a voltage across the output port of the feedback module for connection to the feedback input port of the power converter. This type of feedback module realization is preferably implemented in the first voltage-adding topology described above, in which case the first input node of the feedback module is connected to a node between the power converter and the current regulator, and the second input node of the feedback module is connected to a node between the current regulator and the load. The voltage gain of this type of feedback module can be determined by the ratio of the values of the second and first resistors.

In a further preferred embodiment of the invention, the feedback module can comprise an amplifier without any level-shifting function. Again, the feedback module uses an op-amp to convert a voltage difference across the input port into a voltage across the output port of the feedback module for connection to the feedback input port of the power converter. This type of feedback module realization is preferably implemented in the second voltage-adding topology described above, in which case the first input node of the feedback module is connected to a node between the load and the current regulator, and the second input node of the feedback module is connected to a node common to the current regulator and the power converter, such as the negative supply terminal to the driver circuit.

In some realizations, the feedback voltage setpoint of a switched-mode power converter may already lie within an acceptable range for the voltage drop across the linear current regulator. For example, a commonly used type of commercial switched-mode power converters for battery-powered applications may have a feedback voltage setpoint of about 0.6 V. This lies within the desired range for the voltage drop across the linear current regulator. In this case, the feedback module can preferably be realized to have unity voltage gain. In a particularly straightforward embodiment of the second voltage-adding topology of the invention, the feedback module in such a case may simply comprise a direct connection from the node between current regulator and load, and the feedback input pin of the power converter.

Any suitable converter may be used in the driver according to the invention, for example an off-the-shelf component, whereby it is preferable to use a power-efficient component. An example might be a switched-mode power converter, preferably a synchronous switched-mode power converter. The power converter may be realized as a buck converter when the input voltage supply exceeds the output voltage to be delivered by the power converter. If the input voltage and output voltage ranges overlap, for example if the input voltage might be less than the output voltage to be delivered by the power converter, a combination of boost and buck converter topologies may be implemented, as will be known to the skilled person.

Other objects and features of the present invention will become apparent from the following detailed descriptions considered in conjunction with the accompanying drawings. It is to be understood, however, that the drawings are designed solely for the purposes of illustration and not as a definition of the limits of the invention.

### BRIEF DESCRIPTION OF THE DRAWINGS

FIG. 1 shows a block diagram of a first embodiment of a pulse oximeter arrangement according to the invention;

FIG. 2 shows one embodiment of the feedback module of the driver circuit in the pulse oximeter arrangement of FIG. 1;

FIG. 3 shows an alternative embodiment of the feedback module of the driver circuit in the pulse oximeter arrangement of FIG. 1;

FIG. 4 shows a block diagram of a second embodiment of the pulse oximeter arrangement according to the invention;

FIG. 5 shows an embodiment of the feedback module of the driver circuit in the pulse oximeter arrangement of FIG. 4;

FIG. 6 shows a block diagram of a third embodiment of the pulse oximeter arrangement according to the invention;

FIG. 7 shows waveforms arising during operation of an embodiment of a pulse oximeter arrangement according to the invention;

FIG. 8 shows further waveforms arising during operation of the pulse oximeter arrangement;

FIG. 9 shows absorption spectra of oxygenated haemoglobin and deoxygenated haemoglobin;

FIG. 10 is a block diagram of a prior art pulse oximeter arrangement.

In the drawings, like numbers refer to like objects throughout. Objects in the diagrams are not necessarily drawn to scale.

### DETAILED DESCRIPTION OF THE EMBODIMENTS

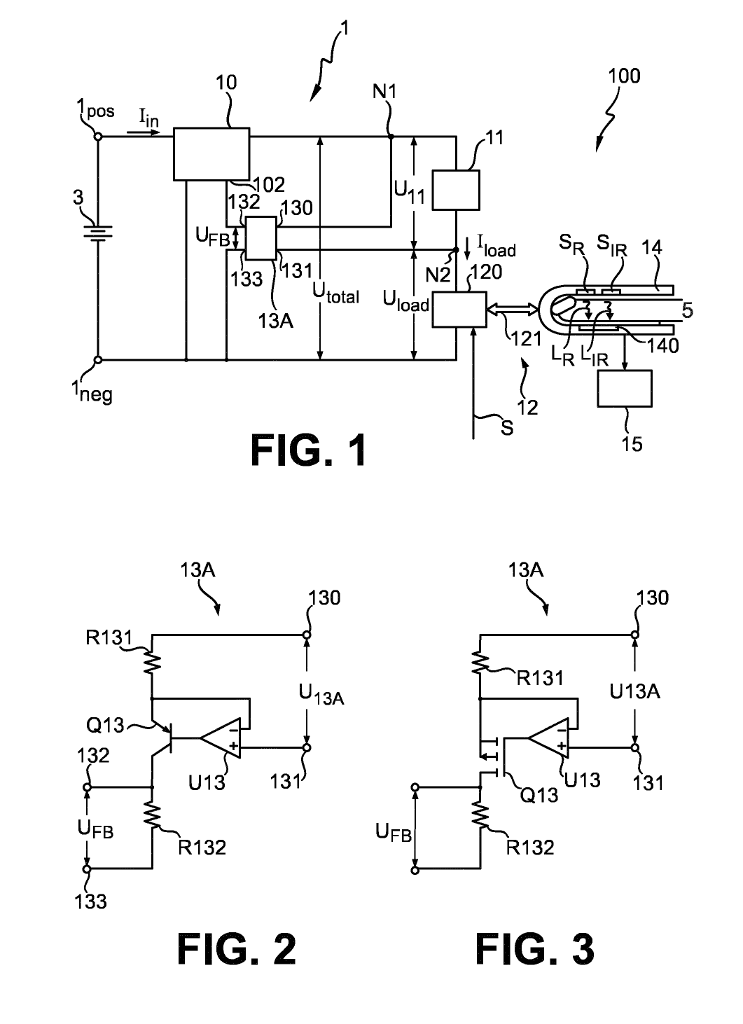

FIG. 1 shows a block diagram of a first embodiment of the inventive driver circuit 1 in a pulse oximeter arrangement 100 according to the invention. A DC power supply 3 to the driver circuit 1 of the pulse oximeter arrangement 100 can comprise a series connection of battery cells, for example two lithium-ion battery cells. The diagram shows a finger hood 14 comprising a red LED light source S_{R} and an infrared LED light source S_{IR}, arranged to fit over a patient's finger 5 for vital signs analysis. Light L_{R}, L_{IR} from a LED light source S_{R}, S_{IR} passes through tissue of the finger 5. Two light paths L_{R}, L_{IR} are shown here, but it should be noted that the LEDs S_{R}, S_{IR} are operated alternately. In this embodiment, a digital control signal S defining the on/off status of the two light sources S_{R}, S_{IR} is input to a switch matrix 120, which generates drive signals for the LEDs S_{R}, S_{IR} accordingly. In this exemplary transmissive oximetry arrangement, a sensor 140 such as a photodetector or photoreceiver arranged on the opposite side of the finger 5 to the LEDs S_{R}, S_{IR} can detect any light that was not absorbed by tissue of the finger 5 and report the measurements to an analysis unit 15. A driver circuit 1 is used to provide a controlled current I_{load} to a load 12 comprising the LED light sources S_{R}, S_{IR} as well as the switch matrix 120 and a connector arrangement 121 that can comprise cables, wires, connectors etc. The driver circuit 1 comprises a power converter 10, a linear current regulator 11, and a feedback module 13A. In this exemplary embodiment, the power converter 10 comprises a DC-DC switched-mode power converter, such as an off-the-shelf component. It may be assumed that the power converter 10 has a buffer capacitor across its output terminals. The power converter output voltage Uₜₒₜₐₗ is the sum of the voltage drop U₁₁ across the linear current regulator 11, and the load voltage U_{load}, measured with reference to a negative supply terminal 1_{neg} of the DC power supply 3. The current output by the linear current regulator 11 is the load current I_{load}, and is essentially the same as the LED current.

As explained above, the forward voltage of an infrared LED is lower than the forward voltage of a red LED. In a pulse oximeter, the red LED S_{R} and the infrared LED S_{IR} are switched in a controlled manner (by the switch matrix 120) such that one of them is only ever “on” when the other is “off”. The driver circuit 1 according to the invention makes use of these facts, and monitors the voltage drop across the current regulator to detect transitions from a high load voltage to a low load voltage (the red LED is turned off and the infrared LED is turned on), and transitions from a low load voltage to a high load voltage (the infrared LED is turned off and the red LED is turned on). To this end, the feedback module 13A in this embodiment comprises a level-shifting amplifier circuit 13A connected between the power converter 10 and the linear current regulator 11 such that a first input 130 node of the feedback module 13A is connected to a node N1 between the power converter 10 and the current regulator 11, and a second input node 131 of the feedback module 13A is connected to a node N2 between the current regulator 11 and the load 12. When the switch matrix 120 turns one of the LED light sources S_{R}, S_{IR} on and the other off, the load voltage U_{load} will tend to either rise or fall accordingly and the same holds for the voltage drop U₁₁ across the current regulator. This transition is detected by the feedback module 13A, and the information is then passed on to the power converter 10, which can adjust its output voltage Uₜₒₜₐₗ accordingly. As a result, the power consumed by the driver circuit 1 is kept to a minimum. This compares favorably with prior art designs, in which the power converter is generally driven to supply a constant output voltage with a rather high value that represents the worst case, not taking into account the difference in forward voltages of the LED light sources S_{R}, S_{IR} and differences in resistive voltage drops across cables, connectors, extension cables etc.

FIG. 2 shows one embodiment of the feedback module 13A of the driver circuit 1 in the pulse oximeter arrangement of FIG. 1. Here, the level-shifting amplifier circuit 13A has an input port comprising a pair of input nodes 130, 131. The differential voltage U_{13A} across the input port 130, 131 is converted to a current using an operational amplifier U13, a transistor Q13 (in this case a PNP bipolar junction transistor), and a resistor R131. The collector or drain current of the transistor Q13 is converted into a voltage U_{FB} across the output port comprising nodes 132, 133 using a further resistor R132. The ratio R132/R131 determines the voltage gain of this amplifier circuit 13A (a current gain β of the transistor

Q13 has negligible influence as long as it is large, e.g. (β>50). For example, a value of 10 kΩ for resistor R131 and a value of 24 kΩ for resistor R132 gives a voltage gain of 2.4. Relatively large resistance values for the resistors R131, R132 ensures that the bias current is negligible with respect to the LED current or load current I_{load}. The required or desired amplifier gain is determined on the basis of the target voltage drop U₁₁ across the current regulator 11 (this can be calculated) and the type of power converter 10 which is to be used (e.g. the setpoint voltage of its feedback input port 101, 1_{neg} has a value determined by the particular power converter chosen). For an embodiment in which the voltage drop U₁₁ across the current regulator 11 is substantially equal to the setpoint voltage of the power converter feedback input port, a voltage gain of unity may be preferred. This can be achieved by selecting equal values for the resistors R131, R132, so that the feedback module 13A acts as a unity-gain level shifter. FIG. 3 shows an alternative version of the feedback module 13A. Here, a unipolar p-type MOSFET Q13 is used instead. Otherwise, the structure and functionality of this version are essentially the same as in the feedback module described in FIG. 2.

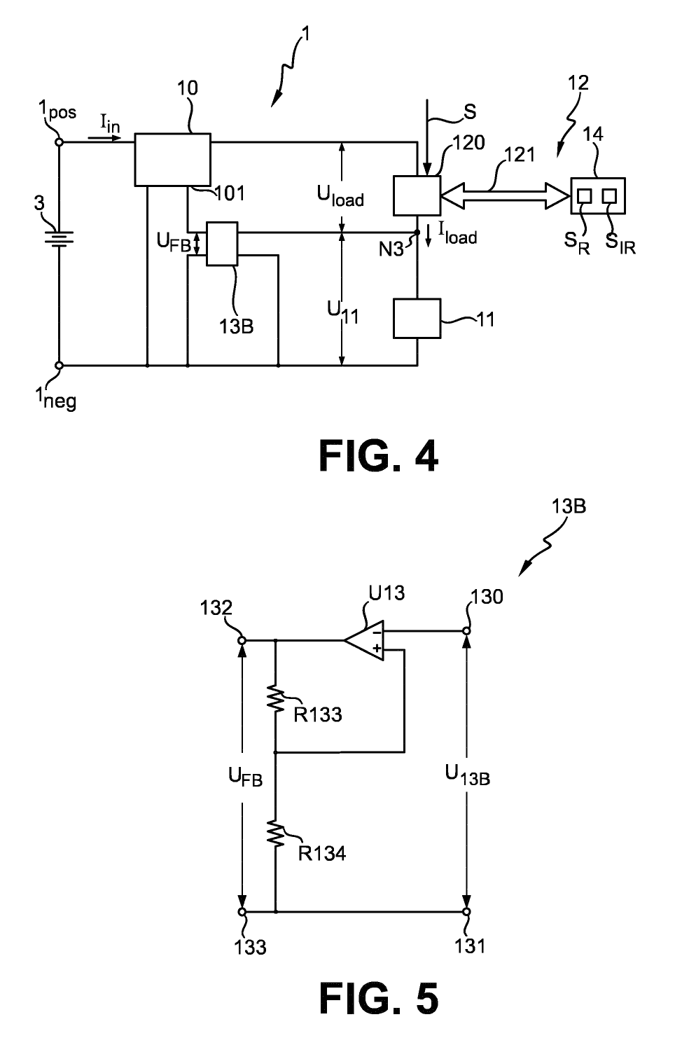

FIG. 4 shows a block diagram of a second embodiment of the driver circuit 1 in a pulse oximeter 100 arrangement according to the invention. The finger hood 14 with its LED light sources S_{R}, S_{IR} is shown in a simplified manner. In this exemplary embodiment, the positions of the load 12 (switch matrix 120, cables and connectors 121, LED light sources

S_{R}, S_{IR}) and current regulator 11 have been interchanged. Such an embodiment may be preferred when the feedback voltage input port 101 of the switched-mode power converter 10 is referenced to the negative supply terminal 1_{neg}. FIG. 5 shows an embodiment of the feedback module 13B in this case. Here, an input port comprising nodes 130, 131 and an output port comprising nodes 132, 133 of the feedback module 13B share a terminal (the negative supply node 1_{neg} in this case). In this way, a level-shifting function is avoided and the feedback module 13B functions as an amplifier with positive voltage gain determined by the resistors R133, R134. An additional advantage of this realization is that an output transistor of the analog current regulator 11 can now be an n-type transistor, e.g. a unipolar NMOS or a bipolar NPN, with the inherent advantages of better performance and lower cost when compared to p-type devices.

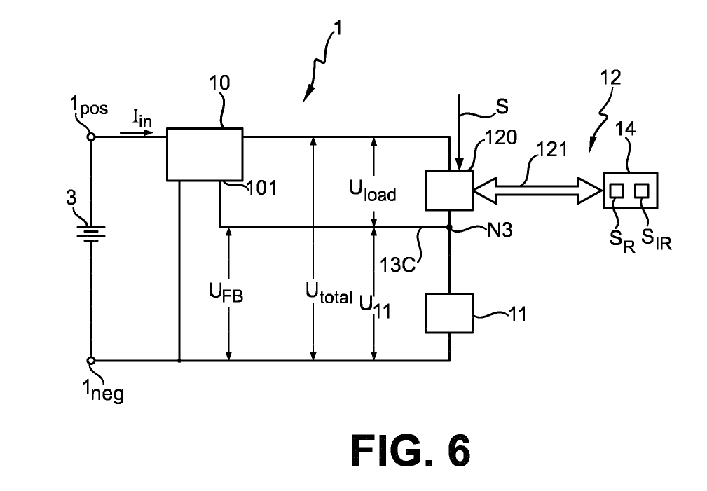

FIG. 6 shows a block diagram of a third embodiment of the driver circuit 1 according to the invention. It shows a variation on the second embodiment of the driver circuit 1 according to the invention. A particular attractive topology results when the voltage gain may be unity. The feedback means 13C may now be implemented by a through connection from node N3 to the feedback input node 101 of the power converter 10, so that the feedback voltage U_{FB} corresponds to the voltage drop U₁₁ across the current regulator 11, measured at node N3. A unity voltage gain is attractive when the setpoint of the feedback voltage U_{FB} of the switched-mode power converter 10 lies in the desired range for the voltage drop U₁₁ across the linear current regulator 11. This may be the case for a voltage feedback setpoint of about 0.6 V, which is a popular value for many off-the-shelf switched-mode power converters designed for use in battery-powered devices.

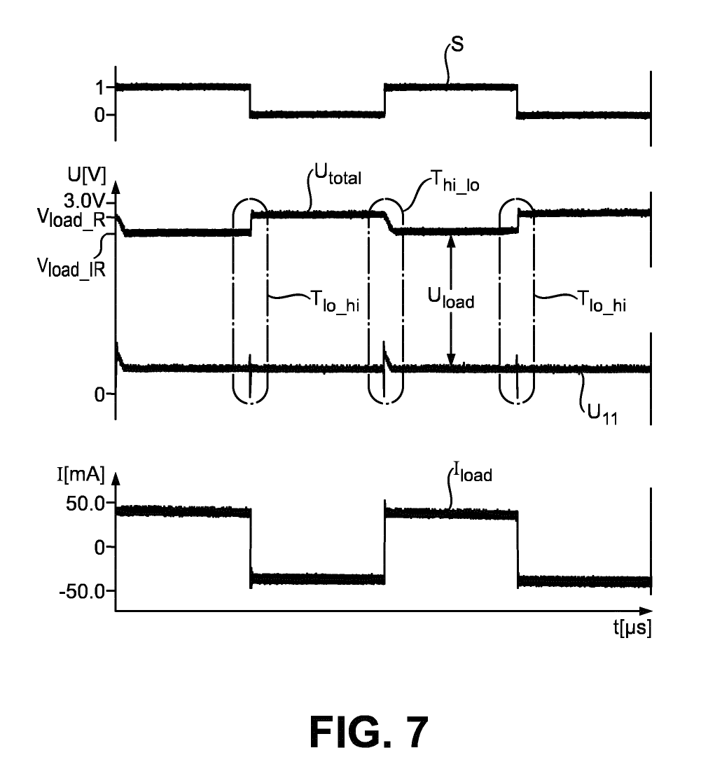

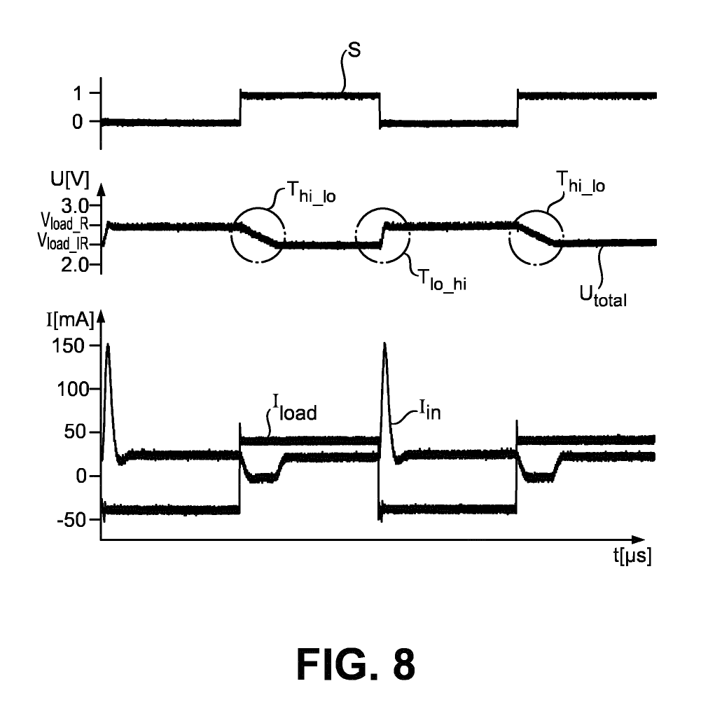

FIGS. 7 and 8 show exemplary waveforms arising during operation of an embodiment of a pulse oximeter arrangement according to the third embodiment of the invention explained above with the aid of FIG. 6. From top to bottom, the diagram shows a digital signal S indicating selection of either red LED (“0”) or infra-red LED (“1”); the output voltage Uₜₒₜₐₗ at the output of the switched-mode buck converter 10; the voltage drop U₁₁ across the linear current regulator 11 (the load voltage U_{load} is the difference between output voltage Uₜₒₜₐₗ and regulator voltage drop U₁₁); and the load current I_{load} as measured through one of two conductors between the switch matrix and the LED load. In these diagrams, the toggling frequency of the LED light sources S_{R}, S_{IR} has a frequency of a few kHz to clearly demonstrate the transitions Tₕᵢ_{_}ₗₒ, Tₗₒ_{_}ₕᵢ or toggling instants. Here, the red and the infra-red light sources S_{R}, S_{IR} are connected anti-parallel, and the current regulator 11 is set to provide a load current I_{load} of 40 mA. The digital signal S is used to control the switch matrix 120 to toggle the LED light sources S_{R}, S_{IR}. Depending on whether the red or the infra-red LED is activated or “on”, the output voltage Uₜₒₜₐₗ of the power converter 10 will settle at a high level V_{load}_{_}_{R} (2.75 V in this case) or a low level V_{load IR} (2.34 V in this case) respectively, owing to the difference in forward voltages of the LED light sources S_{R}, S_{IR}. The voltage drop U₁₁ across the linear current regulator 11 is now identical once more to the voltage reported by the feedback means to the power converter 10, and its steady-state value is 0.6 V in this example.

In FIG. 8, the upper two traces again show the digital select signal S and the output voltage Uₜₒₜₐₗ. The third trace shows the input current Iᵢₙ drawn from an external 5.0 V power supply to the buck converter 10. Its steady-state values are approximately 25 mA and 20 mA, corresponding to the active red LED light source S_{R} and the active infra-red LED light source S_{IR} respectively. The fourth trace again shows the load current I_{load} as measured through one of two conductors between the switch matrix and the LED load. When toggling from the red LED S_{R} to the infra-red LED S_{IR}, the output voltage Uₜₒₜₐₗ of the buck converter 10 drops from the high level V_{load}_{_}_{R} to low level V_{load}_{_}_{IR} in approximately 40 μs in this case. This transition Tₕᵢ_{_}ₗₒ is reported to the power convertor 10 by the feedback means 13C, since the voltage U_{FB} at the output of the feedback means jumps from its steady-state value (0.6V in this case) to a higher value (approximately 1.01 V in this case) directly after the toggle instant, owing to the lower voltage drop across the load. An internal feedback control loop of the DC-DC power converter interprets the higher feedback voltage U_{FB} as a sign to stop delivering energy to its output capacitor. During this transition Tₕᵢ_{_}ₗₒ or voltage ramp Tₕᵢ_{_}ₗₒ, the buck converter 10 allows its output buffer capacitor to partially discharge, and reduces its input current Iᵢₙ to zero during that time. Eventually, depending on the load current and the capacitor value, the feedback voltage U_{FB} reaches its target value (0.6 V in this case) and the output voltage Uₜₒₜₐₗ settles at the low level V_{load}_{_}_{IR}. At that time instant the internal feedback loop of the converter has re-established equilibrium. This is indicated in FIG. 8 as the input current Iᵢₙ of the power converter reaches a new equilibrium level (approximately 20 mA in this case). When toggling from the infra-red LED S_{IR} back to the red LED S_{R}, the feedback voltage U_{FB} drops from its steady-state value of 0.6 V to a much lower value directly after the toggle instant, since the voltage drop across the load increases. The internal feedback control loop of the power converter interprets this as a sign that it should deliver more energy to its output capacitor. This is indicated in FIG. 8 by the short peak on the input current Iᵢₙ at the transition from infra-red to red. As long as the feedback voltage U_{FB} is below its target value, the power converter delivers more energy than required by the load, and the difference is used to rapidly charge the output capacitor. This is accompanied by a sudden brief increase at the power converter's output voltage Uₜₒₜₐₗ. The feedback voltage U_{FB} is increasing at the same time. Shortly afterwards (depending on various parameters such as the maximum output current of the power converter, the load current I_{load} and the output capacitor value) the feedback voltage U_{FB} reaches its target or steady-state value of 0.6V. At that point, the internal feedback loop of the power converter 10 has re-established equilibrium, as indicated by the new equilibrium level (approximately 25 mA in this case) of the input current Iᵢₙ to the power converter 10.

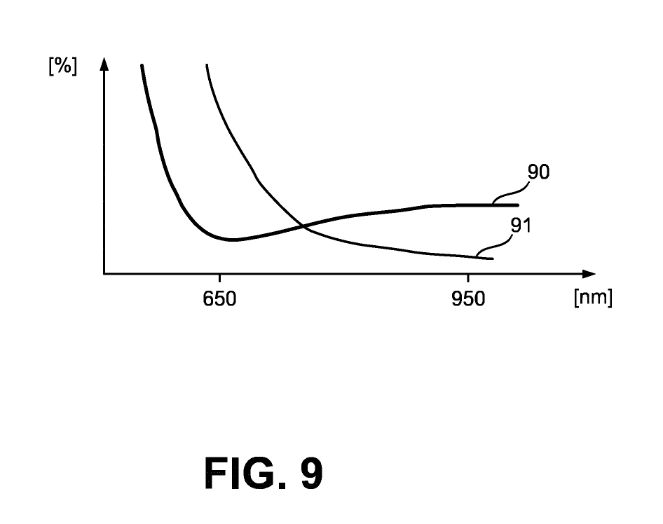

FIG. 9 shows absorption spectra 90, 91 of oxygenated haemoglobin and deoxygenated haemoglobin respectively. At wavelengths of about 650 nm corresponding to red light, deoxygenated haemoglobin absorbs more light than oxygenated haemoglobin. At wavelengths of about 950 nm corresponding to infrared light, oxygenated haemoglobin absorbs more light than deoxygenated haemoglobin. The functional principle of a pulse oximeter is based on these clearly different absorption spectra.

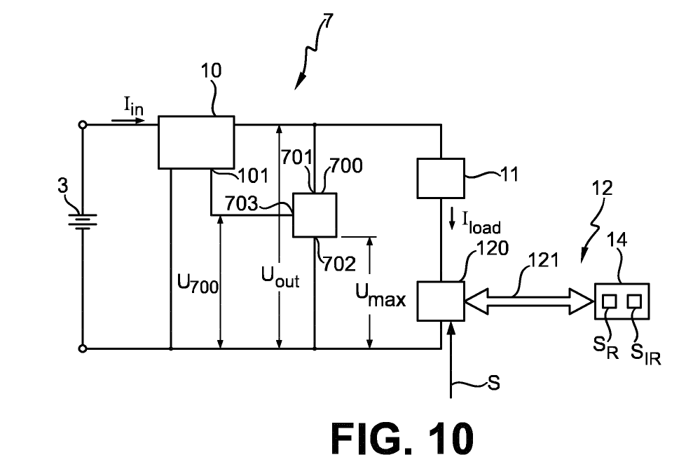

FIG. 10 is a block diagram of a prior art driver circuit 7 in a pulse oximeter arrangement. The setup to a certain extent resembles the embodiments described above. The power converter 10 can be a switched-mode power supply or a low-dropout linear voltage regulator. The feedback loop of the converter 10 is closed in a manner well known to the skilled person by means of a voltage attenuator 700. The input port (nodes 701, 702) of the attenuator 700 is connected to sense the total voltage Uₒᵤₜ at the output of the converter 10. The output port (nodes 703, 702) of the attenuator 700 is connected to the feedback input port of the DC-DC converter to provide the feedback voltage U₇₀₀. The voltage attenuator 700 can be implemented by means of a voltage divider. However, the output voltage Uₒᵤₜ of the power converter 10 is constant in this case, and must at all times be at least as great as the voltage drop across the current regulator 11 plus the maximum voltage drop Uₘₐₓ across the load 12. This is chosen to take into account the highest forward voltage of the LED light sources S_{R}, S_{IR}, as well as any other load resistance. Therefore, the power dissipated by this prior art driver 7 is unnecessarily high, since the power converter 10 must always deliver enough voltage to cover the maximum voltage requirement, irrespective of whether the red LED S_{R} or the infra-red LED S_{IR} is being driven. The prior art realization is therefore characterized by an unfavorably high power dissipation, leading to shorter battery life. Furthermore, since the prior art driver circuit 7 claims a higher voltage margin, there is less leeway in the choice of connectors, cables, and other accessories.

Although the present invention has been disclosed in the form of preferred embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

1. A pulse oximeter driver circuit realized to drive a load comprising at least a red LED light source and an infrared LED light source and a switching module for alternately switching the red LED light source and the infrared LED light source, which driver circuit comprises
a current regulator realized to provide LED current to the LED light sources;
a power converter realized to provide a DC output voltage comprising a current regulator voltage and a load voltage; and
a feedback means realized to obtain a measure of the instantaneous voltage drop across the current regulator and to report the voltage drop measure to the power converter, such that the driver circuit responds to a shift in voltage levels across the load when one LED light source is switched off and the other LED light source is switched on by the switching module.

2. The driver circuit according to claim 1, wherein the feedback means is connected between a feedback input of the power converter and the current regulator.

3. The driver circuit according to claim 1, wherein the feedback means comprises a pair of inputs and a pair of outputs and is realized to convert a voltage difference across the inputs to a feedback voltage across the outputs for connecting to a feedback input of the power converter.

4. The driver circuit according to claim 1, wherein the feedback means comprises an amplifier circuit portion for converting the input voltage difference to the feedback voltage.

5. The driver circuit according to claim 1, wherein a gain of the feedback means is determined on the basis of a target voltage drop across the current regulator.

6. The driver circuit according to claim 1, wherein a gain of the feedback means is determined on the basis of an internal reference setting of the power converter.

7. The driver circuit according to claim 1, wherein the feedback means comprises a level-shifting amplifier.

8. The driver circuit according to claim 1, wherein the feedback means is realized to have unity gain.

9. The driver circuit according to claim 1, wherein the power converter comprises a switched-mode power converter, preferably a synchronous switched-mode power converter.

10. The driver circuit according to claim 9, wherein the power converter comprises a buck converter and/or a boost converter.

11. The driver circuit according to claim 3, wherein the first input of the feedback means is connected to a node between the power converter and the current regulator, and the second input of the feedback means is connected to a node between the current regulator and the load.

12. The driver circuit according to claim 3, wherein the first input of the feedback means is connected to a node between the load and the current regulator, and the second input of the feedback means is connected to a negative supply terminal of the driver circuit.

13. A pulse oximeter arrangement comprising
terminals for connecting to a DC power supply;
a load comprising at least a red LED light source and an infrared LED light source arranged to emit light through human tissue; and
a driver circuit according to claim 1 for driving the load.

14. A method of driving a pulse oximeter load comprising at least a red LED light source and an infrared LED light source and a switching module for alternately switching the red LED light source and the infrared LED light source, which method comprises the steps of
providing a current regulator to deliver drive current to the LED light sources of the load;
providing a power converter to deliver an output voltage comprising an essentially constant voltage drop across the current regulator and a load voltage;
obtaining a measure of the instantaneous voltage drop across the current regulator; and
reporting the voltage drop measure to the power converter for responding to a shift in voltage levels across the load when one LED light source is switched off and the other LED light source is switched on by the switching module.

15. The method according to claim 14, wherein the step of obtaining the voltage drop measure comprises the step of detecting the occurrence of a voltage level transition between a first load voltage level associated with the red LED light source, and a second load voltage level associated with the infrared LED light source.
